(19) United States
(12) Patent Application Publication
(10) Pub. No.: US 2017/0266095 A1
(43) Pub. Date: Sep. 21, 2017

(54) METHOD FOR EXTRACTING AND PRODUCING USEFUL SUBSTANCE, AND SOAP, COSMETICS, OR HEALTH SUPPLEMENT HAVING SAID USEFUL SUBSTANCE AS MAIN COMPONENT

(71) Applicants: Chozo HIRAO, Tokyo (JP); EM COMMUNICATIONS CO., LTD., Higashimurayama-shi, Tokyo (JP)

(72) Inventor: Chozo HIRAO, Tokyo (JP)

(21) Appl. No.: 15/505,472

(22) PCT Filed: Aug. 21, 2015
(86) PCT No.: PCT/JP2015/073458

§ 371 Date: Feb. 21, 2017

(30) Foreign Application Priority Data
Aug. 22, 2014 (JP) 2014-169102

(51) Int. Cl. A61K 8/64; A61Q 19/00; A23L 33/10; C11D 9/40; C11D 9/26; A61K 8/35; A61Q 19/10

(52) U.S. Cl. CPC A61K 8/64; A61K 8/35; A61Q 19/00; A61Q 19/10; C11D 9/40; C11D 9/26; A23L 33/10; A23V 2002/00

(57) ABSTRACT

The present invention simply and efficiently extracts and produces useful substances such as astaxanthin, silk proteins, and polyphenols from a plant material abundantly containing these useful substances. A material such as silk proteins or plant materials containing a useful substance is: immersed in an alkaline aqueous solution which is of a neutral salt and which is obtained by adding a proper amount of an alkaline agent to an aqueous solution of, for example, sodium chloride, calcium chloride, or the like; and reductively decomposed using the reduction potential generated on the alkaline side of the aqueous solution of a neutral salt to extract the useful substance in a state of being dissolved in the aqueous solution of a neutral salt.

### FIELD OF THE INVENTION

The present invention relates to a method for extracting and producing useful substances such as astaxanthin, silk protein, and polyphenol contained in a material through reductive decomposition of the material in a predetermined aqueous solution.

### BACKGROUND OF THE INVENTION

Conventionally, silk fiber such as silk thread and raw silk has been mainly used for clothing. Recently, silk fiber is also used for non-fabric usage of a material of cosmetics, functional food, medical care, and the like. Silk fiber contains two types of silk protein being fibroin (approximately 70%) at the inside and sericin (approximately 30%) at the outside covering fibroin. The two types of silk protein are often separated to be used as an industrial material.

Silk protein has been conventionally used in a clothing field. Recently, owing to that fibroin being a main ingredient of silk fiber contains more than ten types of amino acids, application of silk protein to a non-clothing field is being expanded utilizing such characteristics, especially to soap and cosmetics as focusing on moisture permeability for human skin, natural moisture retaining property, and biocompatibility. Then, focusing on an effect of smoothing one's nerves, an effect of suppressing cholesterol level, and an effect of improving immunity, development of nutritional supplementary food, supplements, and the like for health maintenance is proceeding. Such effects have been proved variously.

Conventionally, separation of silk protein has been performed through processing with an alkaline hydrothermal solution or a method of extracting with high-pressure hot water. For example, a method of refining while controlling silk fiber has been known using an alkaline buffer solution containing an alkaline metal salt made from polybasic acid having a predetermined dissociation constant and an acid such as a boric acid (e.g., see Patent Literatures 1 and 2).

For manufacturing a refined silk fibroin solution for food and drink, a method of dissolving silk fibroin using a hot solution of high concentration calcium chloride has been known (e.g., see Patent Literature 3). In this method, a dialysis-desalting step is required after silk fibroin is dissolved in a salt solution under oxidative atmosphere.

To use sericin obtained from wild silkworm cocoon layers for cosmetics, there has been proposed a method of obtaining a wild silkworm silk sericin solution including steps of refining the wild silkworm cocoon layers as causing the wild silkworm cocoon layers to be contacted with a sodium carbonate solution, still standing, and dialyzing with water (e.g., see Patent Literature 4). The obtained sericin solution is made into skin lotion by adding base ingredients of skin lotion.

Meanwhile, plant materials such as barks and fruits contain a large quantity of polyphenol having superior physiological effects. For manufacturing polyphenol from the plant materials, there have been known various methods such as performing alkali extraction with hot water and adding alkaline metal salt (e.g., Patent Literature 5). Further, there has been proposed a method of obtaining polyphenol-mixed liquor by refining as causing a squeezed or extracted liquid obtained from green fruits such as an apple to pass through an adsorbing agent (e.g., see Patent Literature 6).

### CITATION LIST

### Patent Literature

Patent Literature 1: Japanese Patent No. 1654943
Patent Literature 2: Japanese Patent No. 1882353
Patent Literature 3: Japanese Patent No. 4074923
Patent Literature 4: Japanese Patent Application Laid-open
Patent Literature 5: Japanese Patent Application Laid-open
No. 2009-183229
Patent Literature 6: Japanese Patent No. 3521155

### SUMMARY OF THE INVENTION

### Problem to be Solved by the Invention

When sericin and fibroin are separated through processing with alkaline hydrothermal solution or extracting with high pressure hot water for industrial use of silk protein as described above, in the product, sericin molecules are collapsed to a certain extent, the strength of fibroin as fiber is extremely reduced, the inner structure is collapsed even if the shape as fiber is maintained, and purity is reduced. Accordingly, the product is not preferable for usage of cosmetics, food, medical care, and the like.

In a processing step of cocoons and raw silk, a sericin water is generated in a silk reeling step and an aqueous solution in which 20% or more by weight of the silk is dissolved is generated in a refining step. These have been conventionally disposed as wastes while a legal entity burdens expenses under a specific waste water treatment rule. In a cocoon boiling step to manufacture floss silk, cocoons in which pupas exist or dirty cocoons are boiled with a sodium bicarbonate solution to obtain softness for floss silk. Accordingly, ingredients of pupas are dissolved, so that a sericin solution having a strong odor is generated. Since such a sericin solution has a unique odor, contrivance to extract a useful substance therefrom for usage has not been done thus far.

Conventionally, various reducing agents have been used to dissolve silk fiber. In most cases, when such a reducing agent is turned into an aqueous solution and warmed to accelerate progression of processing, the reducing agent is decomposed and complex decomposition products are extracted therefrom as a result. Accordingly, a processing step is required to eliminate the decomposition products.

It is widely known that polyphenol has an antioxidative effect and a hormonal stimulation effect. It is known that people who frequently eat, for example, fruits having deep coloring matter (e.g., grapes and berries) has a low crisis rate of heart disease such as heart infarction. However, the conventional method with which polyphenol is extracted from a plant material such as bark through boiling with water is extremely inefficient. Accordingly, it has not been put into industrially practical use.

Further, astaxanthin contained in silk protein is known to have a strong antioxidative effect and a physiological effect to protect a living body from ultraviolet light and lipid peroxidation. It is conceivable that astaxanthin is oxidized due to actions of optical energy or oxygen and fibroin molecules in a liquid state are varied to fiber structure due to strong oxidative coupling. Accordingly, to extract astaxanthin from silk protein and to make use of the function thereof, it is desired to reductively solutionize silk protein.

It is conceivable that astaxanthin contained in silk protein is oxidized due to actions of optical energy or oxygen and fibroin molecules in a liquid state are varied to have a fiber structure due to strong oxidative coupling. Accordingly, to extract astaxanthin from silk protein and to make use of the function thereof, it is desired to reductively solutionize silk protein.

To solve the problems of the related art, an object of the present invention is to provide a method to efficiently and easily separate substances of silk protein from silk fiber useful not only for clothing but for non-fabric usage of food, cosmetics, medical care, and the like.

An object of the present invention is to provide a method to efficiently and easily extract useful substances such as silk protein, astaxanthin, and polyphenol from plant materials.

The inventor of the present application has already found that silk protein is typical oxidation-reduction protein.

Further, the inventor of the present application found that astaxanthin which is a type of lipophilicity carotenoide exists in silk protein, and particularly in a sericin II layer. In addition to the strong antioxidative effect, astaxanthin has various physiological activities such as ant inflammatory, antiarteriosclerotic, anti-asthenopia, cerebral function improving, and bloodstream improving.

When silk protein is used as an industrial material for nonfabric usage in the related art, a step of powderizing or solutionizing is generally included. In the powderizing step, since silk fiber is mechanically crushed, progress of oxidization cannot be avoided due to heat accompanying thereat. In the solutionizing step, since silk fiber is hydrolyzed with alkali or acid, solutionizing is performed under oxidative atmosphere. Even when a salting-in method with a high-concentration solution of a neutral salt is adopted to dissolve silk protein while maintaining high molecular weight thereof, processing is performed under oxidative atmosphere as well.

The inventor of the present application performed earnest examination on making use of the characteristic that useful substances such as silk protein and polyphenol are typical oxidation-reduction protein as described above. As a result, a reductive decomposition method was found out to cut coupling of protein molecules structuring silk fiber using reduction potential generated at the alkali side of a neutral salt, that is, reducibility.

The inventor of the present application examined whether polyphenol can be extracted from plant materials with the reductive decomposition method. According to the findings as a result, although polyphenol existing in plant tissues easily cause oxidation polymerization with oxygen and the like in air, polyphenol can be extracted through reduction thereof.

### Means for Solving the Problem

The present invention was executed based on the above findings. That is, a method of extracting useful substances according to the present invention provides a solution containing useful substances through reductive decomposition of a material containing the useful substances performed in an alkaline aqueous solution of a neutral salt.

Since reduction potential is generated at the alkali side even with an alkaline aqueous solution of a neutral salt having relatively low concentration, reductive decomposition of a material can be performed making use thereof and useful substances can be easily dissolved in the solution as extracts.

Further, since reductive decomposition performed in the method of the present invention uses a limited number of types of reagents, reductive decomposition can be performed easily even when dialysis-desalting processing is required for reductive decomposition extracts thereafter.

Since conventional reagents are decomposed when solutionized and warmed, so that complex decomposition products are extracted, processing thereof is extremely difficult. However, the method of reductive decomposition according to the present invention does not have such defect of the related art and brings remarkable profits to various industries in respect of enabling extraction of useful substances through reductive decomposition which has been conventionally impossible.

In one embodiment, the useful substance is astaxanthin. In one embodiment, a material containing a useful substance contains silk protein. Examples of the material containing silk protein include cocoons of domesticated silkworms and wild silkworms, silk threads, raw silk, thrown silk, and raw silk fabric. Here, cocoons include waste cocoons and dirty cocoons.

As described above, in silk protein, a large quantity of astaxanthin is contained mainly in a sericin layer (especially in sericin II). Accordingly, a solution containing astaxanthin in high concentration can be obtained through dissolving the sericin II layer according to the method of the present invention.

In the obtained solution, astaxanthin is exposed to the surface of silk protein molecules in the solution through reductive decomposition. The exposed astaxanthin exerts an antioxidative effect and other effects. For example, a solution containing astaxanthin is suitable to be used as a material of various soaps, cosmetics, bath soap, deodorant, medical cosmetics such as medical cream, food, drink, and supplementary food such as nutrients, and medicine having functions of the antioxidative effect and the like of astaxanthin.

Thus, according to another aspect of the present invention, soap using useful substances extracted with the method of the present invention described above as a material is provided.

In a case that the material is silk protein, depending on the extent of reductive decomposition, useful insolubles can be obtained as reductive decomposition extracts in addition to the solution containing useful substances such as astaxanthin. The insolubles are silk fiber containing a sericin layer which remained without being dissolved at the outer side and fibroin at the inner side. The insolubles can be used for manufacturing silk fiber products.

Accordingly, the method of the present invention can be applied to manufacturing glossed silk as well. For example, when only sericin I at the outermost layer is dissolved through reductive decomposition, astaxanthin having a reductive function is exposed to the surface of sericin II. Accordingly, the obtained glossed silk can exert health functions based on the reductive function.

In another embodiment, a useful substance is polyphenol. In the embodiment, materials containing useful substances are juice extractions, residues of juice extractions, and pericarps of fruits, juice extractions and residues of juice extractions of vegetables, vegetable scraps, fruit thinning of agriculture, and fruit drops. In another embodiment, materials containing useful substances are dicotyledonous plants, especially bowers of dicotyledonous plants such as bark of cedar and white cedar, waste mushroom beds made from wood powder of bowers, or monocotyledonous plants, especially shells or leaves of palm, straw, and squeezed residues of sugarcane. Further, materials containing useful substances include other plant wastes, ferns, and liverworts.

Since a large quantity of polyphenol exists, in the plant materials, in an oxidation polymerization state due to air, a solution containing polyphenol at high concentration can be obtained through reductive decomposition of the plant materials performed in an alkaline aqueous solution of a neutral salt. Further, although many of the plant materials have been conventionally disposed as waste, effective use of resources can be attempted with the present invention.

For example, in a case that the plant materials are food or something that was food such as juice extractions, residues of juice extractions, or pericarps of fruits, juice extractions or residues of juice extractions of vegetables, vegetable scraps, fruit thinning of agriculture, or fruit drops, the obtained polyphenol solution is suitable to be used as materials of additives of food or drink and health food. In a case that the plant materials are bark or waste mushroom beds of dicotyledonous plants, monocotyledonous plants, ferns, or liverworts, the obtained polyphenol solution is suitable to be used as materials of pharmacon such as deodorant for environment, deodorant, surface treatment agent of coating material, and static electricity control agent.

The inventor of the present application found a characteristic that reduction potential generated at the alkali side of an aqueous solution of a neutral salt becomes stronger as the temperature of the aqueous solution gets higher. In one embodiment, reductive decomposition of a material is performed with the temperature of an aqueous solution of a neutral salt being between room temperature and around 120° C.

Further in another embodiment, useful substances can be extracted to a solution more effectively through performing reductive decomposition of a material under high pressure.

In another embodiment, the alkaline aqueous solution of a neutral salt is acquired by adding an alkaline agent to a water-soluble neutral salt that is acquired by combining any one as a positive ion selected from calcium, magnesium, aluminium, sodium, potassium, ammonium, and amins, and any one as a negative ion selected from hydrochloric acid, acetic acid, citric acid, oxalic acid, formic acid, boric acid, nitric acid, and sulfuric acid. Such an alkaline aqueous solution of a neutral salt shows various reductive functions in accordance with the combination of a neutral salt and an alkaline agent. Accordingly, reductive decomposition can be performed under an optimum condition by selecting an appropriate alkaline agent in accordance with a useful substance to extract and/or a material.

In one embodiment, the neutral salt is sodium chloride or calcium chloride and the alkaline agent is sodium carbonate anhydrous or sodium hydroxide. Such an alkaline aqueous solution of a neutral salt is especially preferable in a case that reductive decomposition is performed while maintaining high molecular weight with silk protein being the material.

### BRIEF DESCRIPTION OF THE DRAWINGS

FIG. 1 illustrates variations of oxidation-reduction potentials when 0.1 mol aqueous solution of caustic soda is added to 0.1 mol solution of a neutral salt.

FIG. 2 illustrates variations of oxidation-reduction potentials when 0.1 mol crystallized neutral salt is added to 0.1 mol aqueous solution of caustic soda of 100 ml.

FIG. 3 illustrates variations of reduction potentials when liquid temperature is increased in cases, which are a part of FIG. 2, that the added neutral salt is sodium chloride and trisodium citrate.

### DETAILED DESCRIPTION OF THE PREFERRED EMBODIMENT

In the following, methods for extracting a useful substance according to the present invention will be described in detail using preferable embodiments.

According to a first embodiment of the present invention, having silk fiber containing silk protein as a material, useful substances contained therein can be extracted. The silk fiber is dissolved in a predetermined alkaline aqueous solution of a neutral salt using reduction action thereof.

As described above, silk protein is oxidation-reduction protein in which protein molecules are coupled through oxidation-reduction reaction. Accordingly, silk protein can be dissolved without being collapsed through dissociation of oxidative coupling of protein molecules due to reduction action of the alkaline aqueous solution of a neutral salt.

The useful substances contained in silk protein are extracted to the solution or exposed to the surface of the silk protein molecules in the solution in an activated state. For example, when sericin I at the outermost layer and sericin II being the second layer are dissolved among sericin layers of silk protein, astaxanthin is exposed to the surface of sericin II molecules in the solution in a state of being capable of exerting the antioxidant effect. Although effects of astaxanthin have been conventionally exerted in a state of being incorporated in the structure of silk protein, it has become possible to derive the superior function at maximum by exposing astaxanthin to the surface of the silk protein molecules.

The whole amount of the material is not necessarily dissolved. Insolubles are extracted depending on the extent of dissolving. However, in a case that the material is silk protein, other useful substances being usable are still contained in the remaining insolubles. When such useful substances are exposed to the surface of the silk protein molecules in the insolubles in an activated state, the function thereof can be exerted by using silk protein as silk protein. Further, the useful substances can be isolated by performing further treatment on the insolubles.

For example, when only sericin I at the outermost layer is dissolved among sericin layers of silk protein, astaxanthin is exposed to the surface of sericin II molecules of silk protein in the obtained insolubles in a state of being capable of exerting the antioxidative effect as well. Thus, the superior function of astaxanthin may be derived at maximum as well. Since astaxanthin is ester bond to sericin II, astaxanthin can be separated therefrom by being heated in an acid solution.

For materials of silk protein, any silk fiber may be used, for example, including cocoons of domesticated silkworms and wild silk worms, silk threads, raw silk, spun silk yarn, thrown silk, silk wastes, and raw silk fabric. Waste cocoons and dirty cocoons which have been conventionally disposed may be used as the cocoons as well.

The alkaline aqueous solution of a neutral salt may be adjusted by adding an alkaline agent to a solution of a neutral salt. The neutral salt is a water-soluble salt acquired by combining any one as a positive ion selected from calcium, magnesium, aluminium, sodium, potassium, ammonium, amins, and the like, and any one as a negative ion selected from hydrochloric acid, acetic acid, citric acid, oxalic acid, formic acid, boric acid, nitric acid, sulfuric acid, and the like.

By combining the positive ion and negative ion variously, various kinds of neutral salts can be prepared.

The combination may be appropriately selected in accordance with the purpose of use of reductive decomposition extracts, conditions of reductive decomposition, and the like. Specifically, considering reductive decomposition of silk protein, a salt composed of a univalent metal such as sodium or potassium is preferable.

Alternatively, a salt composed of a divalent metal may be used to make use of characteristics thereof. Further, a volatile neutral salt may be chosen so that excessive reagents are easily eliminated from extracts after reductive decomposition of a material is performed. Considering the object of the present invention, it is preferable that substances that cause environmental pollution or generate toxic materials are eliminated in advance.

The alkaline aqueous solution of a neutral salt exerts various reductive functions depending on the combination of a neutral salt and alkaline agent. Accordingly, the alkaline agent is appropriately selected in consideration with combination of a neutral salt to be used so that an optimum reductive decomposition condition is obtained corresponding to the useful substance to extract and/or a material. For example, in a case that the neutral salt is sodium chloride or calcium chloride, sodium carbonate anhydrous or sodium hydroxide can be used as the alkaline agent.

Even if the alkaline aqueous solution of a neutral salt has low concentration, strong reducibility is shown owing to a reductive potential generated at the alkaline side. For example, an aqueous solution having concentration of 0.015% obtained by mixing sodium carbonate anhydrous against sodium chloride (2:1) shows strong reducibility as the oxidation-reduction potential E is −214 mV. Since such an aqueous solution shows further strong reducibility when boiled, strong reducibility can be stably maintained by keeping the aqueous solution boiled for a required processing time.

The Inventor of the present application found a phenomenon that an aqueous solution of a neutral salt has a peculiar oxidation-reduction potential and the oxidation-reduction potential moves to the reduction potential side when turned into alkaline, and that the reduction potential increases with increase of the solution temperature, so that strong reduction action is shown. The present invention utilizes the phenomenon for industrial manufacturing.

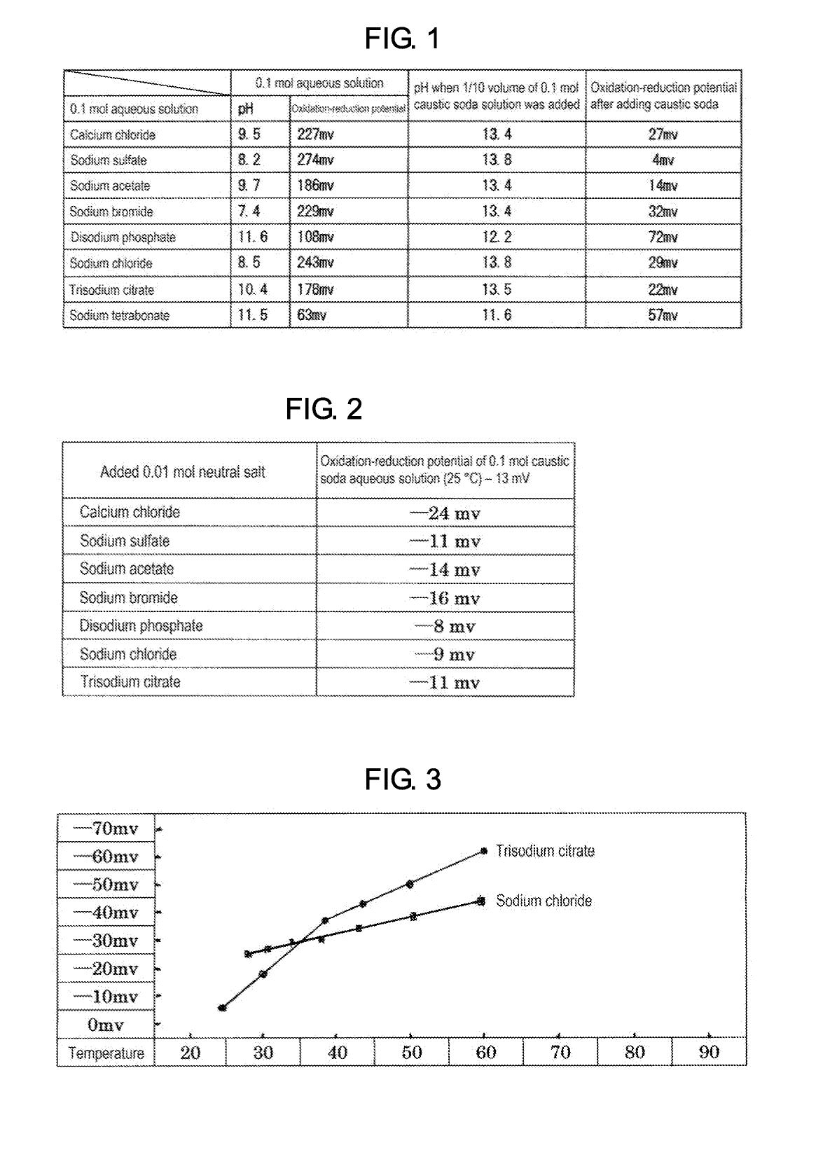

FIG. 1 illustrates measured variations of oxidation-reduction potentials when 0.1 mol aqueous solution of caustic soda is added to 0.1 mol solution of a neutral salt . It can be acknowledged that, in a case of a neutral salt having large buffer capacity, variation of the potential is small owing to that variation width toward alkali is small.

FIG. 2 illustrates measured variations of oxidation-reduction potentials when 0.1 mol crystallized neutral salt is added to 0.1 mol aqueous solution of caustic soda of 100 ml.

FIG. 3 illustrates measured variations of reduction potentials when the solution temperature is increased in cases, which are a part of FIG. 2, that the added neutral salt is sodium chloride or trisodium citrate. Although measurement was only performed up to 60° C. owing to restriction due to functions of measurement instruments, the graph illustrated in FIG. 3 shows that the reduction potential increases as the solution temperature increases.

Here, in the present application, measurement was performed, as a supplementary examination, until the solution temperature fallen to room temperature after the solution was boiled for ten minutes to estimate stability of the reduction potential at temperature regions where measurement cannot be performed. As a result, the reduction potential roughly returned to the potential at the initial state. This is an important characteristic that indicates that alkaline reductive decomposition can be utilized as industrial technology.

A low concentration alkaline aqueous solution of a neutral salt composed of mixture of sodium chloride and sodium carbonate anhydrous at such ratio is suitable for reductive decomposition processing of raw silk. For example, by performing processing on the mixture for a predetermined time in an aqueous solution of 1% of raw silk and liquor ratio being 25 to 40 under a substantially boiling state or temperature near 95° C, the raw silk can be dissolved. The extent of dissolving can be understood by a feeling by touching the raw silk with a hand during processing, for example.

An amount of the alkaline agent to add to the aqueous solution of a neutral salt can be adjusted in accordance with a type and condition of the silk fiber to be used as the material, object, and the like. For example, in a case that the material is wild silkworms, swelling state of silk fiber is adjusted by adjusting the amount of sodium carbonate anhydrous as well as the case of domesticated silkworms, so that desired reductive decomposition processing can be performed. In a case that the material is wild raw silk, the natural green color that the raw silk holds by nature can be kept by proceeding the reductive decomposition processing at an approximately neutral region.

For example, in a case that an alkaline aqueous solution of a neutral salt containing sodium citrate and sodium carbonate anhydrous at a ratio of four to one, silk protein of the material is completely separated into fibroin and sericin by boiling the material for two hours. Especially, a solution of silk protein having reductive decomposition performed in an alkaline aqueous solution of a neutral salt containing sodium citrate and high-concentration alkaline agent has strong reducibility, so that it is suitable to be used as a base material of functional soap having the antioxidant effect.

According to the method of the present invention, since intermolecular coupling of silk protein is dissociated with reduction action and protein is not collapsed, separating and extracting can be performed while preventing strength of fibroin to be remarkably reduced and maintaining required activity of sericin. Accordingly, the both can be used as it is as industrial material having high purity.

It is verified that the solution of silk protein molecules obtained in the present embodiment has a strong coating formation function. Accordingly, using the solution as a material of soap, cosmetics, and bath additives, the coating formation function effectively acts to protect skin, and further, the reductive function of the silk protein, that is, the antioxidative effect effectively delays aging of skin.

In a case that reductive decomposition of silk fiber is performed in an alkaline aqueous solution of a neutral salt in which an alkaline agent is added to a 50% solution of calcium chloride, not only the sericin layer at the outer side but also the fibroin at the inner side can be completely dissolved due to the strong reduction potential. It is known that liquefied silk protein has functions of deodorization, electrostatic control, and the like in addition to the abovementioned antioxidative effect due to functions of the various useful substances contained therein. Making use of the functions, silk protein can be used as a base material of goods using the functions and effects for various usages such as cosmetics, medicine, deodorization, suppressing static electricity, and surface treatment.

For manufacturing the base material for such industrial usage, it is required that reductive decomposition processing of materials is performed more effectively. The reducibility of the alkaline aqueous solution of a neutral salt is strengthened through being pressurized at about 1.2 atmospheric pressure. As a result, a solution containing desired reductive decomposition extracts at a higher concentration can be obtained, so that application for wider usage can be enabled.

For example, in a case that reductive decomposition of silk fiber is performed in an alkaline aqueous solution of a neutral salt in which 0.1% sodium chloride is added to 10% hot solution of sodium hydroxide under high pressure of 1.2 atmospheric pressure, a silk protein solution having high concentration of 20% can be obtained. The oxidation-reduction potential E when the solution is neutralized is −400 mV showing strong reducibility. The reducibility is a sufficient function as a base material for manufacturing medicine, for example.

As described above, a sericin water generated in the silk reeling step and an aqueous solution in which 20% or more by weight of the silk is dissolved in the refining step, in the processing step of cocoons and raw silk, have been disposed as industrial wastes and the unique odor has hampered reuse thereof. According to the method of the present invention, such sericin solution can be used as a material as well. In such a case, reductive decomposition is performed on the odor as well by adding an appropriate amount of an alkaline aqueous solution of a neutral salt to the sericin solution. Accordingly, an odorless extract, that is, sericin solution is obtained.

In a cocoon boiling step to manufacture floss silk, cocoons in which pupas exist or dirty cocoons are boiled with a sodium bicarbonate solution to obtain softness for floss silk. Accordingly, ingredients of pupas are dissolved, so that a sericin solution having a strong odor is generated. According to the method of the present invention, such sericin solution can be used as a material as well. In this case, an appropriate amount of an alkaline aqueous solution of a neutral salt is added to the sericin solution and boiling processing is performed for five minutes. Then, chrysalis oil which is a main causative agent of the odor being separated from sericin in the solution is eliminated through filtration. Thus, an odorless extract, that is, sericin solution is obtained.

In either case, the amount of the alkaline aqueous solution of a neutral salt to be added to the sericin solution is sufficient in the order of 0.02 to 0.06% of the amount of the sericin solution being the material. Owing to the reductive decomposition processing, oxidative coupling of the silk protein molecules contained in the material is dissociated and useful substances such as astaxanthin and lipid contained in silk protein are exposed to the surface of the sericin molecules. Accordingly, the obtained extract of the sericin solution can be used as a base material for various industrial usage described above.

According to a second embodiment of the present invention, polyphenol which is a useful substance contained in a plant material is extracted to a solution through reductive decomposition. Thus, polyphenol is extracted. Examples of the plant material include juice extractions, residues of juice extractions, pericarps, and fruit drops of various fruits such as apples, mandarins, berries such as grapes, and pineapples, juice extractions and residues of juice extractions of various vegetables, vegetable scraps, and fruit thinning of agriculture. Examples of the plant material further include dicotyledonous plants, especially bowers of dicotyledonous plants such as bark of cedar and white cedar, waste mushroom beds made from wood powder of bowers, monocotyledonous plants, especially shells and leaves of palm and workpieces thereof, straw, and squeezed residues of sugarcane, other various plant wastes, ferns, and liverworts.

Such plant materials contain a large quantity of polyphenol. However, since most of the polyphenol exist in an oxidation polymerization state among molecules due to air, the polyphenol has not been generally used so far. According to the present embodiment, oxidation polymerization among the polyphenol molecules can be cut through reductive decomposition processing of the plant materials in a predetermined alkaline aqueous solution of a neutral salt. Thus, a solution containing polyphenol in a usable state at high concentration can be easily obtained.

The obtained polyphenol solution can be used for various usages. For example, in a case that the plant materials are food or something that was food such as juice extractions, residues of juice extractions, and pericarps of fruits, juice extractions and residues of juice extractions of vegetables, vegetable scraps, fruit thinning of agriculture, fruit drops, shells of palm, and workpiece thereof, the obtained polyphenol solution is suitable to be used as materials of additives of food or drink and health food. In a case that the plant materials are bark, waste mushroom beds, shell of palm, or workpiece thereof, the obtained polyphenol solution is suitable to be used as a material of pharmacon such as deodorant for human body (oral cavity) and environment, deodorant, surface treatment agent of coating material, and static electricity control agent. Thus, according to the present embodiment, plant materials that have been disposed as wastes can be effectively used as resources.

In the above, preferable embodiments of the present invention are described in detail. However, the present invention is not limited to the above embodiments and those skilled in the art will appreciate that various modification may be applied.

### FIRST EXAMPLE

Having refined cocoons (cocoons from which sericin I is eliminated through refining) as a material, astaxanthin and phosphorus lipid contained therein was extracted to a solution through reductive decomposition.

Concretely, 1 kg of refined cocoons was added to 4 litters of alkaline aqueous solution of a neutral salt in which 2 kg of calcium chloride anhydrous and 40 g of sodium carbonate anhydrous are dissolved. Then, the refined cocoons were dissolved through ting while maintaining temperature at about 90° C. Adding of cocoons was performed in four times in equal amounts. The dissolving processing was performed for 30 minutes each and two hours in total.

It was verified with color reaction that the obtained solution contained astaxanthin which shows grayish blue color with ester bond with sericin. Further, from study results of the inventor of the present application, it is estimated that the obtained solution contains phosphatidylserine, phosphatidylcholine, and phosphatidylethanolamine which exist in sericin II layer of silk protein.

Thus, the refined cocoons were dissolved and astaxanthin and the abovementioned three types of phosphorus lipid were extracted to the solution. This solution has been disposed as waste liquid without being used in the conventional refining step of silk.

### SECOND EXAMPLE

Having cut cocoons not being refined as a material, astaxanthin and phosphorus lipid contained therein was extracted to a solution through reductive decomposition.

Concretely, 0.8 kg of squashed cut cocoons were added to 4 litters of alkaline aqueous solution of a neutral salt in which 2.66 kg of calcium chloride dihydrate and 4 g of sodium hydroxide are dissolved. Then, the squashed cut cocoons were dissolved through agitating while maintaining temperature at about 75 to 80° C. It became possible to freely agitate the cut cocoons in the solution one hour after being added to the solution and dissolving processing was performed through agitating for about 30 minutes thereafter. Since the dissolved solution had extremely high concentration and high viscosity, 4 litters of water was added to perform filtration.

Similarly to the above, it was verified with color reaction that the obtained solution contained astaxanthin which shows grayish blue color with ester bond with sericin. Further, it is estimated that the obtained solution contains phosphatidylserine, phosphatidylcholine, and phosphatidylethanolamine which exist in the sericin II layer of silk protein.

Thus, the cocoons not being refined were dissolved and astaxanthin and the abovementioned three types of phosphorus lipid were extracted to the solution. Further, silk protein such as sericin is dissolved in the solution. Through gelatification, for example, after performing dialysis-desalting with water, the silk protein can be used for various usages as described above.

### THIRD EXAMPLE

Having refined silk yarns (obtained by eliminating sericin I at the outermost layer through refining raw silk) as a material, astaxanthin and the like contained therein was extracted to a solution through reductive decomposition.

Concretely, 1 kg of refined silk yarns were added to 4 litters of alkaline aqueous solution of a neutral salt in which 2.66 kg of calcium chloride dihydrate and 40 g of sodium carbonate anhydrous are dissolved. Then, the refined silk yarns were dissolved through agitating while maintaining temperature at about 80 to 90° C.

Insolubles were eliminated through filtration or centrifugation. It was verified with color reaction that the obtained solution contained astaxanthin which shows grayish blue color with ester bond with sericin.

### FOURTH EXAMPLE

Having refined silk yarns (obtained by eliminating sericin I at the outermost layer through refining raw silk) as a material, astaxanthin and the like contained therein was extracted to a solution through reductive decomposition.

Concretely, 24 kg of refined silk yarns were added to an alkaline solution of a neutral salt in which 1 g of sodium carbonate anhydrous and 50 g of calcium chloride anhydrous are dissolved in 100 ml of water. Then, the refined silk yarns were dissolved through agitating while maintaining temperature at about 85° C. Under the same dissolving condition, 50 g of refined silk yarns were dissolved with the calcium chloride anhydrous replaced to 100 g.

In either cases, astaxanthin was contained in the obtained solution.

### FIFTH EXAMPLE

Having silk yarns as a material, astaxanthin and the like contained therein was extracted to a solution through reductive decomposition. Soap was manufactured using the solution.

Concretely, 5.5 g of silk yarns and 2 g of granular charcoal (millimeter order) were added to an alkaline aqueous solution of a neutral salt in which 3 g of citric acid and 112 g of potassium hydroxide are dissolved in 250 ml of purified water. Then, the silk yarns were dissolved through agitating while maintaining temperature at about 80° C. After being naturally cooled to room temperature, insolubles were eliminated through filtration. Then, purified water was further added to the obtained solution and the total weight was adjusted to 530 g. Then, 150 g of coconut oil and 375 g of olive oil were added, agitation was performed for about ten minutes under temperature at about 45° C. to turn into an emulsion state, and agitation was additionally performed for about five minutes while heating until starting to slightly boil. Thus, soap of about 1 kg was manufactured.

### SIXTH EXAMPLE

Having silk wastes (bisu) as a material, astaxanthin and the like contained therein was extracted to a solution through reductive decomposition. Soap was manufactured using the solution.

Concretely, 13 g of silk wastes (bisu) and 6 g of granular charcoal (millimeter order) was added to an alkaline aqueous solution of a neutral salt in which 10 g of citric acid and 330 g of sodium hydroxide are dissolved in 1.16 litter of purified water. Then, the silk wastes were dissolved through agitating while maintaining temperature at about 80° C. After being naturally cooled to room temperature, insolubles were eliminated through filtration. Then, purified water was further added to the obtained solution and the total weight was adjusted to 1550 g. Then, 60 g of stearin acid, 600 g of coconut fatty acid, and 1500 g of olive oil were added and agitation was performed while heating until turning into a transparent oily state. Thus, soap of about 3.6 kg was manufactured.

### SEVENTH EXAMPLE

Having residues of apple juice as a material, polyphenol contained therein was extracted to a solution through reductive decomposition.

Concretely, the residues of apple juice were added to an alkaline aqueous solution of a neutral salt in which 0.01 mol sodium chloride is dissolved in 0.2 to 0.3 mol sodium hydroxide aqueous solution being ten times of the residues of apple juice in volume. Then, boiling decomposition was performed for five to ten hours while the solution was kept refluxed, and polyphenol was extracted. Sequentially, insolubles were eliminated through filtration, so that an alkaline aqueous solution containing the extracted polyphenol was obtained. The obtained alkaline aqueous solution can be used as it is or after removing alkali with ion exchange resin depending on usage.

### EIGHTH EXAMPLE

Having waste mushroom beds as a material, polyphenol contained therein was extracted to a solution through reductive decomposition. Concretely, the waste mushroom beds were added to a solution in which 0.01 mol sodium chloride is dissolved in 0.5 to 1 mol sodium hydroxide aqueous solution being ten to twenty times of the waste mushroom beds in volume. Then, boiling decomposition was performed for five to ten hours while the solution was kept refluxed, and polyphenol was extracted. Sequentially, insolubles were eliminated through filtration, so that an alkaline aqueous solution containing the extracted polyphenol was obtained. The obtained alkaline aqueous solution can be used as it is or after neutralizing with hydrochloric acid depending on usage.

### NINTH EXAMPLE

Having residues of shells of coconut after fiber parts thereof are extracted as a material, polyphenol contained therein was extracted to a solution through reductive decomposition.

Concretely, 1 kg of soil conditioner (product name: Cocopeat (registered trademark)) made of residues of coconut shells was added to an alkaline aqueous solution of a neutral salt in which 200 g of sodium dithionite, 200 g of sodium hydroxide, and 0.5 g of sodium chloride are dissolved in 50 litters of water. Then, boiling decomposition was performed for six hours while the solution was kept refluxed, and polyphenol was extracted. Sequentially, insolubles were eliminated through filtration, so that an alkaline aqueous solution containing the extracted polyphenol was obtained. Since the solution showed high alkalinity of pH 11.7, the solution was neutralized to pH 8 by adding a small quantity of concentrated hydrochloric acid.

Content of polyphenol contained in Cocopeat which is the material is about 53%. Accordingly, it is estimated that about 500 g of polyphenol are contained in the obtained alkaline aqueous solution.

### TENTH EXAMPLE

Having straw representing monocotyledonous plants as a material, polyphenol contained therein was extracted to a solution through reductive decomposition. Processing conditions of reductive decomposition is the same with the ninth example.

Concretely, 1 kg of straw was added to an alkaline aqueous solution of a neutral salt in which 200 g of sodium dithionite, 200 g of sodium hydroxide, and 0.5 g of sodium chloride are dissolved in 50 litters of water. Then boiling was performed while the solution was kept refluxed, and polyphenol was extracted. Sequentially, insolubles were eliminated through filtration, so that an alkaline aqueous solution containing the extracted polyphenol was obtained.

Useful substances were extracted to the obtained alkaline aqueous solution for about 53% of the material in weight ratio. Considering an assumption amount of alkali in the reductive decomposition processing of the present example, it is estimated that the extracted useful substances are mixtures of polysaccharide and polyphenol. Accordingly, the aqueous solution is suitable for materials of health food using, for example, a protection function of the extracted useful substances.

### ELEVENTH EXAMPLE

Having liverworts as a material, polyphenol contained therein was extracted to a solution through reductive decomposition. Processing conditions of reductive decomposition is the same with the ninth example as well.

Concretely, 1 kg of liverworts were added to an alkaline aqueous solution of a neutral salt in which 200 g of sodium dithionite, 200 g of sodium hydroxide, and 0.5 g of sodium chloride are dissolved in 50 litters of water. Then boiling was performed while the solution was kept refluxed, and polyphenol was extracted. Sequentially, insolubles were eliminated through filtration, so that an alkaline aqueous solution containing the extracted polyphenol was obtained.

Polyphenol was extracted to the obtained alkaline aqueous solution for about 40% of the material in weight ratio. An assumption amount of alkali in the reductive decomposition processing of the present example was the same extent with the tenth example.

1.-12.(canceled)

13. A method of extracting a useful substance comprising:
obtaining a solution containing astaxanthin through reductive decomposition of a material containing astaxanthin performed in an alkaline aqueous solution of a neutral salt acquired by adding an alkaline agent to a water-soluble neutral salt that is acquired by combining any one as a positive ion selected from calcium, magnesium, aluminium, sodium, potassium, ammonium, and amins, and any one as a negative ion selected from hydrochloric acid, acetic acid, citric acid, oxalic acid, formic acid, boric acid, nitric acid, and sulfuric acid.

14. A method of extracting a useful substance comprising:
obtaining a solution containing silk protein through reductive decomposition of a material containing silk protein performed in an alkaline aqueous solution of a neutral salt acquired by adding an alkaline agent to a water-soluble neutral salt that is acquired by combining any one as a positive ion selected from calcium, magnesium, aluminium, sodium, potassium, ammonium, and amins, and any one as a negative ion selected from hydrochloric acid, acetic acid, citric acid, oxalic acid, formic acid, boric acid, nitric acid, and sulfuric acid.

15. The method of extracting a useful substance according to claim 13,
wherein the material containing the useful substance is cocoons of domesticated silkworms or wild silkworms, silk threads, raw silk, thrown silk, or raw silk, the cocoons including waste cocoons and dirty cocoons.

16. The method of extracting a useful substance according to claim 13, wherein the neutral salt is sodium chloride or calcium chloride and the alkaline agent is sodium carbonate anhydrous or sodium hydroxide.

17. The method of extracting a useful substance according to claim 16,
wherein the reductive decomposition is performed in the alkaline aqueous solution of a neutral salt at a temperature equal to or higher than room temperature and not higher than around 120° C.

18. The method of extracting a useful substance according to claim 17,
wherein the reductive decomposition is performed under 1.2 atmospheric pressure.

19. Soap, cosmetics, or nutrition supplement containing a useful substance extracted with the method according to claim 13.

20. Soap, cosmetics, or nutrition supplement containing a useful substance extracted with the method according to claim 14.
